(19) United States
(12) Patent Application Publication
(10) Pub. No.: US 2017/0182244 A1
(43) Pub. Date: Jun. 29, 2017

(54) INFUSION PUMP LINE IDENTIFICATION

(71) Applicant: Smiths Medical ASD, Inc., Plymouth, MN (US)

(72) Inventor: Michael BLOMQUIST, Plymouth, MN (US)

(73) Assignee: Smiths Medical ASD, Inc., Plymouth, MN (US)

(21) Appl. No.: 15/325,199

(22) PCT Filed: Jul. 8, 2015
(86) PCT No.: PCT/US2015/039477

§ 371 Date: Jan. 10, 2017

Related U.S. Application Data
(60) Provisional application No. 62/025,214, filed on Jul. 16, 2014.

(51) Int. Cl. A61M 5/168; A61M 5/14; A61M 39/28; A61M 5/142

(52) U.S. Cl. CPC A61M 5/16859; A61M 5/14228; A61M 5/1407; A61M 39/28; A61M 5/16827; A61M 2005/16868; A61M 2005/16872; A61M 2205/18; A61M 2205/60

(57) ABSTRACT

A method identifies to which one of a plurality of infusion pumps one of a plurality of fluid lines is coupled. The method can include intentionally producing a predetermined pressure pattern in one of the plurality of fluid lines, detecting the predetermined pressure pattern by way of a sensor of one of the plurality of infusion pumps, and indicating detection of the predetermined pressure pattern in the one of the plurality of fluid lines, thereby indicating the one of the plurality of infusion pumps to which the one of the plurality of fluid lines is coupled. In some cases, a tool configured to occlude and the squeeze the fluid line can be used to intentionally produce the predetermined pressure pattern.

### TECHNICAL FIELD

This disclosure relates to infusion pumps, and more particularly, to multiple infusion pumps correspondingly coupled to multiple fluid lines.

### BACKGROUND

Infusion pumps are useful medical devices for providing medicaments to patients. For example, medications such as antibiotics, chemotherapy drugs, and pain relievers are commonly delivered to patients via infusion pumps, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe, enteral, and insulin pumps. Infusion pumps are typically useful in various routes of medicament delivery, including intravenously, intra-arterially, subcutaneously, intraperitoneally, in close proximity to nerves, and into an intraoperative site, epidural space or subarachnoid space.

When multiple infusion pumps are used to deliver medicaments to an individual, it can be difficult to determine by visual inspection which tube(s) or line(s) (as termed throughout this disclosure, “line” or “lines”) are connected to each pump. In some patient populations, for example, it is a common clinical scenario to have six to nine infusion pumps in use at one time to provide therapy to a single patient, with use of one or two dozen infusion pumps for a single patient being not unheard-of. As known to medical practitioners, harm to the patient or even death can result if the wrong medicament is infused at the wrong infusion site. For example, if antibiotics are infused into an epidural catheter, serious harm to the patient can result. In addition to preventing wrong connections, correct line-to-pump identification may help prevent wrong line disconnections (such as from a manifold), with the attendant sterility issues that arise with line disconnections and subsequent connections.

In view of the importance of correct line-to-pump association, there is a need for improved systems and methods for properly identifying which lines are coupled to which infusion pumps.

### SUMMARY

This disclosure relates to infusion pumps, and more particularly, to multiple infusion pumps correspondingly coupled to multiple fluid lines.

In an illustrative but non-limiting example, the disclosure provides a method for identifying to which one of a plurality of infusion pumps one of a plurality of fluid lines is coupled. The method can include intentionally producing a predetermined pressure pattern in one of a plurality of fluid lines, detecting the predetermined pressure pattern by way of a sensor of one of a plurality of infusion pumps, and indicating detection of the predetermined pressure pattern in the one of the plurality of fluid lines, thereby indicating the one of the plurality of infusion pumps to which the one of the plurality of fluid lines is coupled. Intentionally producing the predetermined pressure pattern can further include occluding the one of the plurality of fluid lines at a first location, and while occluded at the first location, squeezing the one of the plurality of fluid lines. With regard to the squeezing, this can include squeezing between the first location and the one of the plurality of infusion pumps. The squeezing can include squeezing the one of the plurality of fluid lines at least two times.

In some cases, the intentionally producing the predetermined pressure pattern can include actuating a tool configured to perform the occluding and the squeezing. In some instances, the tool can be configured to be readily engaged with and disengaged from any of the plurality of fluid lines when the lines of the plurality of fluid lines are deployed for use, where the tool, when engaged, is capable of performing the occluding and the squeezing. In some other instances, the tool can be configured to be not readily disengageable from the one of the plurality of fluid lines when the fluid line is deployed for use. The tool can be structured to perform the occluding and the squeezing sequentially as handles of the tool are progressively brought together. The tool can be configured to reversibly lock after occluding the one of the plurality of fluid lines. The tool can be configured to reversibly lock after the occluding and the squeezing of the one of the plurality of fluid lines. Alternately, the tool can be configured not to lock after the squeezing of the one of the plurality of fluid lines, thereby permitting squeezing of the line multiple times without locking interference by repeatedly tightening and relaxing a hold on the tool.

When the method includes the use of a tool to occlude and squeeze to produce a predetermined pressure pattern, the method can further include monitoring for a release pressure pattern associated with release of the tool. In such cases, the method can also include either or both of maintaining an indication of detection of the predetermined pressure pattern if the release pressure pattern has not been detected; and/or annunciating an alarm if a pre-determined condition is met and the release pressure pattern has not been detected.

In some cases, the indicator can be disposed in or on the one of the plurality of infusion pumps. In some cases, the sensor can be configured to perform upstream occlusion detection. In some cases the sensor can be configured to perform downstream occlusion detection. In some cases, the sensor can be disposed downstream relative to a valve of the one of the plurality of fluid lines.

In illustrative examples of the present disclosure, a predetermined pressure pattern can be defined as a relationship of pressure vs. time in a fluid line that is attributable to intentionally producing the relationship, with such intentional production being more complex than creation of a single occlusion of the fluid line.

In another illustrative but non-limiting example, the disclosure provides a system for confirming that a fluid line is connected to an infusion pump. The system can include a fluid line, an infusion pump configured to pump fluid in the fluid line, and a tool configured to engage the fluid line. The infusion pump can include a sensor configured to detect pressure of fluid in the fluid line and output information related to the pressure of the fluid, a controller operatively coupled to the sensor, and an indicator operatively coupled to the controller. The tool can be configured to, when engaged with the fluid line, both occlude the fluid line and subsequently squeeze the fluid line, where the actions of occluding and squeezing the fluid line produce a predetermined pressure pattern in the fluid line. The controller of the infusion pump can be programmed and configured to receive the information related to the pressure of the fluid, interpret the information related to the pressure of the fluid to recognize the predetermined pressure pattern, and if the predetermined pressure pattern is recognized, provide an indication via the indicator.

In yet another illustrative but non-limiting example, the disclosure provides an infusion pump that can include a pumping mechanism configured to supply a fluid medicament from a reservoir to a patient via a line, a sensor configured to measure pressure of the fluid medicament in the line and output information related to the pressure, a controller operatively coupled to the sensor, and an indicator operatively coupled to the controller. The controller can be programmed and configured to receive the information related to the pressure, interpret the information related to the pressure to recognize any of a group of one or more predetermined pressure patterns, and if any of the group of one or more predetermined pressure patterns is recognized, provide an indication via the indicator.

In still another illustrative but non-limiting example, the disclosure provides a device for creating a predetermined pressure pattern in a fluid line. The device can include a clamp portion configured to occlude the fluid line, one or more squeeze portions, with each of the one or more squeeze portions configured to squeeze the fluid line and thereby produce a pressure pulse in the fluid line, and a grip mechanically connected to the clamp portion and the one or more squeeze portions. The grip can be configured and connected such that as the grip is actuated, the clamp occludes the fluid line and then each of the one or more squeeze portions squeeze the fluid line sequentially. In some cases, the clamp occludes, and then each of the one or more squeeze portions squeeze, the fluid line sequentially as the grip is actuated in a continuous one-way motion. The device can further include a lock mechanism configured to reversibly lock after the clamp portion occludes the fluid line such that the fluid line remains occluded by the clamp portion. Alternatively or in addition, the device can further include a lock mechanism configured to reversibly lock after the one or more squeeze portions squeeze the fluid line such that the fluid line remains squeezed by the one or more squeeze portions. In some cases, the device can be structured such that it does not necessarily lock after the one or more squeeze portions squeeze the fluid line.

In still yet another illustrative but non-limiting example, the disclosure provides a method for identifying an infusion pump to which one of a plurality of fluid lines is coupled. The method can include intentionally producing a predetermined pressure pattern in one of a plurality of fluid lines, detecting the predetermined pressure pattern by way of a sensor of an infusion pump, and indicating detection of the predetermined pressure pattern in the one of the plurality of fluid lines, thereby indicating that the one of the plurality of fluid lines is coupled to the infusion pump.

The above summary is not intended to describe each and every example or every implementation of the disclosure. The description that follows more particularly exemplifies various illustrative embodiments.

### BRIEF DESCRIPTION OF THE FIGURES

The following description should be read with reference to the drawings. The drawings, which are not necessarily to scale, depict several examples and are not intended to limit the scope of the disclosure. The disclosure may be more completely understood in consideration of the following description with respect to various examples in connection with the accompanying drawings, in which:

FIG. 1 is a schematic illustration of several infusion pumps being used to provide therapy to a single patient;

FIG. 2 is a schematic illustration of an example of an infusion system that includes line identification features;

FIG. 3 is a graph of pressure vs. time that may be exhibited in a downstream portion of a fluid line connected to a running pump;

FIG. 4 is a graph of pressure vs. time that may be exhibited in a fluid line that is repeatedly squeezed and released;

FIG. 5 is a graph of pressure vs. time that may be exhibited in a fluid line that is occluded at a first location, then repeatedly squeezed and released at another location while remaining occluded at the first location;

FIG. 6 is a graph of pressure vs. time that may be exhibited in a fluid line that is occluded at a first location, then manipulated to produce ramped pulses;

FIG. 7 is a graph of pressure vs. time that may be exhibited in a fluid line that is manipulated with a tool to produce a predetermined pressure pattern, after which the tool may produce a release pressure pattern associated with release of the tool;

FIG. 8 is a schematic diagram of portions of a syringe infusion pump system;

FIG. 9 is a schematic diagram of portions of a syringe infusion pump system that includes additional features that may improve monitoring of pressure in the fluid line.

### DESCRIPTION

The following description should be read with reference to the drawings, in which like elements in different drawings may be numbered in like fashion. The drawings, which are not necessarily to scale, depict selected examples and are not intended to limit the scope of the disclosure. Although examples of construction, dimensions, and materials may be illustrated for the various elements, those skilled in the art will recognize that the examples provided may have suitable alternatives that can be utilized.

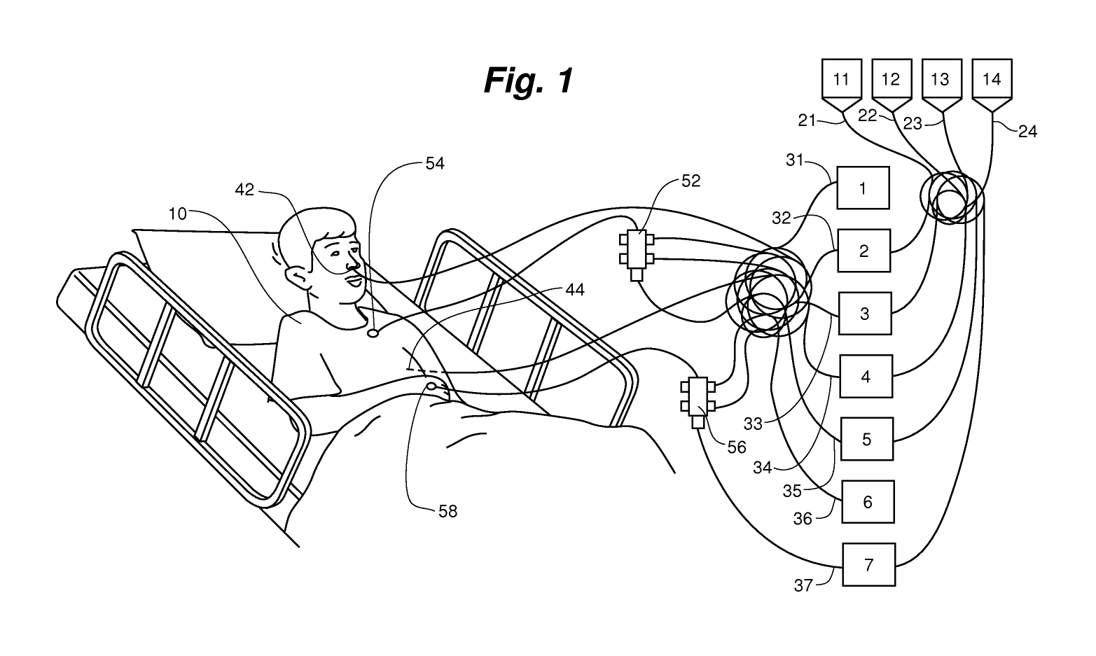

FIG. 1 is a schematic illustration of an example of several infusion pumps 1-7 being used to provide therapy to a single patient 10. Since any suitable infusion pumps can be used, they are represented schematically in FIG. 1. In this example, pumps 2, 3, 5, 7 can draw their respective medicaments from external reservoirs 11-14, which can, for example, be conventional IV (intravenous) bags, or any other suitable reservoirs. Pumps 1, 4, and 6 can draw their respective medicaments from internal or closely integrated reservoirs such as cassettes, syringes, or other any other suitable reservoirs.

Each pump 1-7 can be operatively connected to one or more fluid lines, which can be, for example, upstream lines 21-24, as shown in the drawing, through which medicaments can be supplied from respective reservoirs 11-14 to pumps, and/or downstream lines 31-37 through which medicaments can be delivered from respective pumps 1-7 to the patient 10. Some of lines 31-37 can transport medicaments essentially directly to an infusion site such as enteral infusion site 42 and epidural infusion site 44. Some of lines 31-37 can transport medicaments to an infusion manifold such as manifold 52 which delivers to inter-arterial infusion site 54 and manifold 56 which delivers to intra-venous infusion site 58.

In FIG. 1, upstream lines 21-24 and downstream lines 31-37 are intentionally drawn such that it can be difficult to visually trace a line between a pump and a reservoir, between a pump and a manifold, between a pump and an infusion site, or generally, between a pump and a distal (relative to the pump) location on the line. In a real-world setting, the lines generally would not be purposely arranged so as to make such visual traces difficult, but such difficulties may indeed arise as a consequence of the complexities of multi-pump and multi-line infusions. Even in situations in which appropriate care is taken to keep track of lines and their respective connections, it can still be difficult to associate any given line with a pump to which it is connected. As discussed elsewhere, the consequences of wrong connections in medicament delivery can be dire, and thus it would be decidedly advantageous to provide systems and methods that can assist medical practitioners and other authorized users in associating lines with pumps. As will be further described, this disclosure provides such systems and methods.

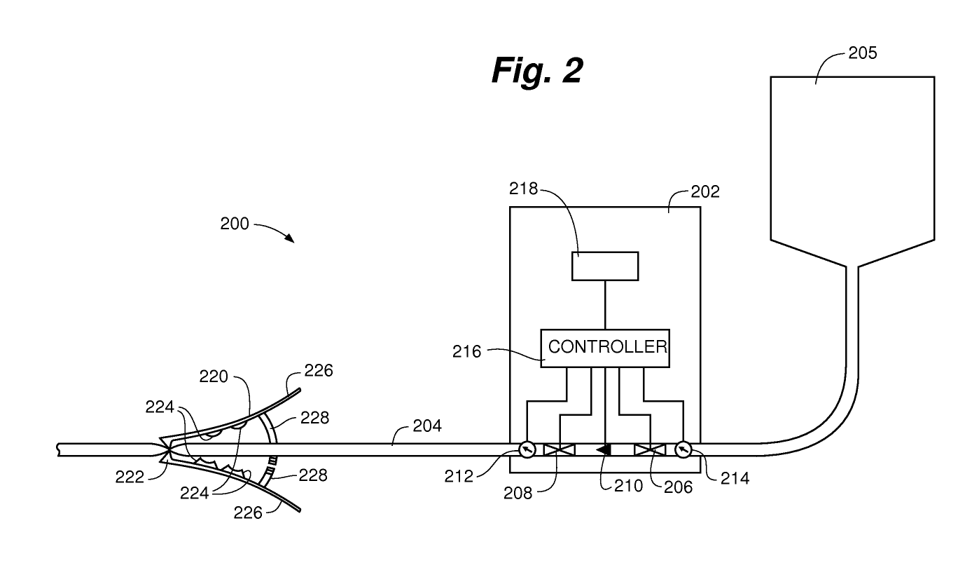

FIG. 2 is a schematic illustration of an example of an infusion system 200 that includes line identification features (and a tool 220 as will be further described). Infusion system 200 includes an infusion pump 202 that is configured to pump fluid in fluid line 204 that is operatively coupled to the pump. Fluid in line 204 can flow downstream toward a patient for administration, for example toward the left side of FIG. 2. Fluid can feed into line 204 from an upstream reservoir 205, which is illustrated in FIG. 2 as an external reservoir such an IV bag, but this is just one example of a medicament reservoir. As described with respect to FIG. 1, reservoirs alternatively can be located in or on pumps.

Infusion pump 202 can include one or more valves, such as upstream valve 206 and downstream valve 208; however, this valve arrangement is merely an example and is not necessary for all implementations contemplated in the present disclosure. Infusion pump 202 can include a pump mechanism, symbolically represented at 210 in FIG. 2. Any appropriate pump mechanism 210 can be employed. The location of the symbol at 210 in FIG. 2 should not be considered limiting as to the location of the pump mechanism, nor should the designation of reference numeral 210 be interpreted as excluding any other numbered features of pump 202 from participating in the pump mechanism. For example, in some instances, one or both of valves 206, 208 can work in concert with an expulsor, which can be located between the valves (e.g., approximately where symbol 210 is located in FIG. 2), to provide a peristaltic-type pump mechanism.

Infusion pump 202 can include one or more sensors 212, 214 configured to measure pressure of fluid in the fluid line 204. Sensor 212 can be a downstream sensor, and sensor 214 can be an upstream sensor. Sensors 212 and 214 can be configured to output information related to the pressure of the fluid, for example, to a controller 216 of pump 202. Sensor 212 can be a so-called “downstream occlusion” (DSO) sensor that is configured to perform downstream occlusion detection, but this is not necessary. In some examples, sensor 212 can be a downstream sensor provided separately from a DSO sensor, and in some examples, pump 202 may not include a DSO sensor. Analogously, sensor 214 can be, but is not necessarily, a so-called “upstream occlusion” (USO) sensor. Lines drawn in FIG. 2 between controller 216 and other features of pump 202, such as valves 206, 208 and pump mechanism 210, indicate operative couplings that may exist between the controller and the features. Pump 202 can also include an indicator 218 disposed in or on the pump and operatively coupled to controller 216. Some examples of an infusion system 200 can include an indicator physically separate from pump 202, as discussed further elsewhere herein. Any suitable indicator 218 or indicators can be used. Such indicators can be visual, audio, tactile, digital-signal-based, or based upon any mode(s) of communication that can be employed to indicate a status of pump 202 and its associated external components such as fluid line 204. In some examples, an indicator or indicators can be provided in coordination with and/or as part of a user interface system of an infusion pump and/or infusion system.

The present disclosure contemplates the use of pressure sensors such as, for example, one or more of sensors 212 and 214 to assist in the association of one or more fluid lines, such as lines 21-24 and 31-37 of FIG. 1, and particular pumps to which they can be connected or coupled, such as pumps 1-7. In an illustrative example (Scenario A), a plurality of pumps such as pumps 1-7 of FIG. 1 may be known infusion pumps each featuring a DSO sensor and associated occlusion alarm system. (Note that in the present disclosure, all described scenarios are merely illustrative in nature and no representation is made that the scenarios have been physically implemented in actuality, unless otherwise stated explicitly.) In example Scenario A, all pumps can be actively pumping medicaments over time (“running”) respectively. A medical practitioner or authorized user who wants or needs to identify or verify a pump to which a corresponding infusion line is attached can intentionally and temporarily occlude a line (with a clamp, or by manual pinching, for example) and then wait for an occlusion alarm to be indicated on one of the running pumps (for example, via indicator 218), after which it can be deduced, with some degree of certainty, that the line that was so intentionally and temporarily occluded is connected to the particular pump that indicated the alarm.

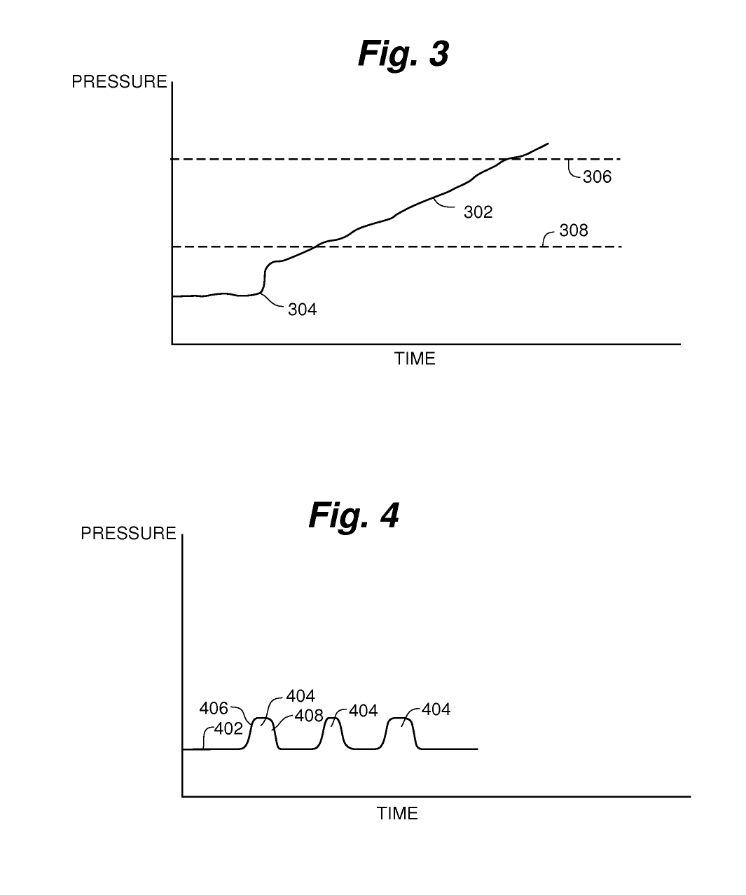

FIG. 3 is a graph of pressure vs. time that may be exhibited in a downstream portion of a fluid line connected to running pump as in Scenario A. A signal is represented by 302 in the drawing, and is related to pressure in a fluid line, as might be output from a sensor such as sensor 212 of FIG. 2. At 304, the line may be intentionally occluded. Prior to the occlusion (to the left of 304), the pressure may be substantially constant. At the time of the occlusion, the pressure may increase rapidly, at least in part because of the portion of fluid displaced by the action (e.g., pinching, clamping, etc.) that causes the occlusion. Depending on the length of the line affected by the action, a lesser or a greater portion of fluid may be displaced, with a relatively smaller or larger pressure increase. After the occlusion (to the right of 304), the pressure may rise gradually as the pump continues to work to pump fluid in the occluded portion of the line. The representation in the graph of the pressure rise to the right of 304 is merely schematic, and the particular profile taken may vary according to the particular occurrences. For example, in some cases a running pump can deliver pulses of medicament separated by intervals without fluid delivery, which might produce a “stair-step” pressure rise profile (not illustrated) in comparison with the relatively continuous upward slope of FIG. 3. The rate of pressure rise due to pumping can be relatively gradual compared to the rapid pressure increase at the time of the occlusion at 304. When the pressure signal (302) rises above an occlusion alarm threshold value 306, occlusion detection logic (implemented, for example, in controller 216) can responsively trigger an occlusion alarm, for example via indicator 218. Because the pressure rise due to pumping may be relatively gradual, the time elapsed between the occlusion at 304 and the triggering of the alarm (after the signal represented by 302 crosses the occlusion alarm threshold value 306) can be substantial. In some instances, a threshold value 308 lower than occlusion alarm threshold value 306 can be implemented such that an indication can be provided when the signal represented by 302 crosses the lower threshold value 308. Such an indication based upon lower threshold value 308 can allow a user to identify which line is related to which pump after a shorter time interval compared solely to relying upon an occlusion alarm corresponding to higher threshold value 306. Although not illustrated, it is to be appreciated and understood that a similar scenario could exist when an upstream line is occluded (e.g., close to reservoir 205 of FIG. 2). In such a case, the pressure in the line may increase initially as the line is occluded, then slowly decrease (rather than increase, as in FIG. 3) as the pump attempts to draw fluid from the occluded line (or, as the pump “draws vacuum”). Appropriate thresholds for indicating when such a situation occurs or is suspected of occurring can be established, as will be apparent to those of skill in the art.

While the example of Scenario A can be useful in associating fluid lines with pumps, there can be a number of aspects that could be improved. For example, the time delay between intentional line occlusion and indication of the alarm can be substantial, possibly depending on a rate of fluid delivery in that line and other physical characteristics. Another potential shortcoming is the possibility of misinterpreting an occlusion alarm for an unintentionally occluded line as an alarm due to an intentional occlusion for line identification, thereby potentially leading to line misidentification. Another possible drawback is that it may not be possible to identify a line with the sequence of Scenario A (i.e., intentionally occluding a line, then waiting for an increase in pressure as the pump continues to run) on a line that is already occluded, where a user might need or want to identify which of many lines is unintentionally occluded. Scenario A also would not be expected to identify a line attached to a pump that is idle, or that is otherwise not actively delivering medicament, since pressure changes owing to medicament pumping would not be present, and further in such an instance the occlusion sensor(s) and/or indicator may not be operative.

In view of these and other considerations, the present disclosure contemplates systems and methods to produce predetermined pressure patterns in fluid lines, and detecting said predetermined pressure patterns, which can assist in the association and identification of the fluid lines with pumps. A predetermined pressure pattern can be defined as a relationship of pressure vs. time in a fluid line that is attributable to the deliberate action of an agent to produce the relationship, where the deliberate action is more complex than creation of a single occlusion of the fluid line. The deliberate action can create a predetermined series or sequence of pressure changes in a fluid line that are detectable by a pressure sensor.

In an illustrative example (Scenario B), a medical practitioner or other appropriate agent (e.g., clinician, caregiver, user, robot, or any other suitable entity) can squeeze and release an infusion line repeatedly in any appropriate manner (e.g., by hand or with a tool). FIG. 4 is a graph of pressure vs. time that may be exhibited in the infusion line in this scenario. Reference numeral 402 represents a signal related to pressure in a fluid line. A number of pressure pulses 404 can be observed, each corresponding to a squeeze and release of the line. For each pulse 404, a rapid increase in pressure 406 associated with the squeezing of the line may be followed by a rapid drop in pressure 408 associated with the release of the line. In the present disclosure, a “squeeze” of a line may occlude the line, but this is not necessary. A “squeeze” may narrow or otherwise alter the shape of the inner bore (where fluid resides) of a line sufficiently to create a pressure change in the line.

In this example (Scenario B) of a predetermined pressure pattern, as in at least some other predetermined pressure patterns of the present disclosure, there is at least one segment during which the pressure decreases rapidly after having risen rapidly. The relative term “rapidly” can be considered with respect to more gradual pressure changes that can be caused in an infusion line by pumping in or on an occluded line. Also in this example of a predetermined pressure pattern, as in at least some other predetermined pressure patterns of the present disclosure, there are at least two separate segments during which the pressure increases rapidly, although this is not required.

The pressure pattern signal 402 of FIG. 4 can be detected by an infusion pump such as, for example, infusion pump 202 of FIG. 2. One or more of sensors 212, 214 can measure pressure of fluid in the fluid line and output information related to the pressure of the fluid. Controller 216 can be programmed and configured to receive the information related to the pressure of the fluid and to interpret the information related to the pressure of the fluid to recognize the predetermined pressure pattern. If the predetermined pressure pattern is recognized, controller 216 can be programmed and configured to provide such an indication via the indicator of the infusion pump 202. In some descriptions, the measurement of pressure information combined with interpretation and recognition of a predetermined pressure pattern can be collectively described as “detecting a predetermined pressure pattern.” An act of detecting a predetermined pressure pattern can be performed by any appropriate component or combination of components of infusion pump 202, such as one or more of sensors 212, 214 in combination with controller 216 as described. In some examples, a sensor subsystem of a pump can be capable of performing the act of detecting a predetermined pressure pattern without involvement of a central pump controller such as controller 216. In this disclosure, the act of detecting a predetermined pressure pattern can be described as being performed by a subset of the components involved in said act, such as “the sensor” or “the controller,” which should not be construed as excluding other components from involvement in the act.

In some examples, a predetermined pressure pattern can be detected by components that are not necessarily integral to or built into an infusion pump. For example, an accessory device capable of sensing pressure in a line and recognizing pressure patterns could be reversibly and selectively attached to a line attached to an infusion pump proximal the infusion pump. In another example, an infusion pump or other device could transmit pressure sensor information to an external device such as a server, a monitor, another pump, or any other suitable device, which could be configured to interpret the information to recognize the predetermined pressure pattern. When a predetermined pressure pattern is recognized, whether it be recognized by pump components or an external device, an indication of such can be provided in any appropriate manner via any appropriate indicator. In addition or as an alternative to an indicator on the pump to which the line in which the predetermined pressure pattern was recognized is attached, it is contemplated that an indication could be provided on a “dashboard” or control panel that can, for example, provide status information for a system of multiple infusion pumps. Such a dashboard can be provided on a device physically separate from the pump. Providing an indication of detection of a predetermined pressure pattern could also include transmitting an information signal, for example, via a hospital information network. These are just some examples.

Detecting the predetermined pressure pattern of Scenario B/FIG. 4 can involve recognizing more sophisticated patterns of pressure vs. time as compared with Scenario A, in which an indication may be triggered relatively simply by a pressure reaching a threshold value. Any suitable interpretation and recognition methods and algorithms can be employed. For example, the pattern of FIG. 4 could be recognized by an alternating sequence of upward and downward pressure transitions meeting defined criteria, such as transitioning through (upwardly and/or downwardly) one or more pressure values. Another example is comparing a measured pattern with a previously recorded pattern and creating a similarity score. In some cases, a measured pattern can be “fit” (via linear regression, for example) to a mathematical model, and if fit constants are within certain ranges, recognition of the pattern could be declared. Any appropriate filtering (low-pass, high-pass, band-pass, etc.) can be performed on data as part of the interpretation/recognition algorithm. In some cases, for example, a running pump with an occluded line might exhibit a slowly-varying rise in pressure upon which a rapidly-varying predetermined pressure pattern can be overlaid, whereas if the same rapidly-varying predetermined pressure pattern is produced in an idle system, the slowly-varying component may not exist. A high-pass filter can be employed to filter out the nearly-DC slow-varying component, and recognition can be performed on the surviving AC signal. These are just some examples of aspects of interpretation and recognition methods, and any appropriate methods can be used.

Production and detection of a predetermined pressure pattern like that of Scenario B and FIG. 4 for line association/identification purposes can feature a number of advantages. The pump connected to the fluid line does not need to be running (but may be running) for the pressure pattern to be produced in the line and recognized by the sensor/controller. The shape of the pressure patterns might differ in some aspects with the pump running or not running, but it is expected that significant aspects of the pressure patterns would be the same. For example, the slopes of the “plateaus” between rapid increases 406 and drops 408 in pressure may differ, but the “lands” between pulses 404 and the sloped portions 406, 408 may be largely alike in the running vs. non-running cases. In contrast, some pressure patterns may rely upon pumping action of the pump to vary the pressure vs. time in an expected manner.

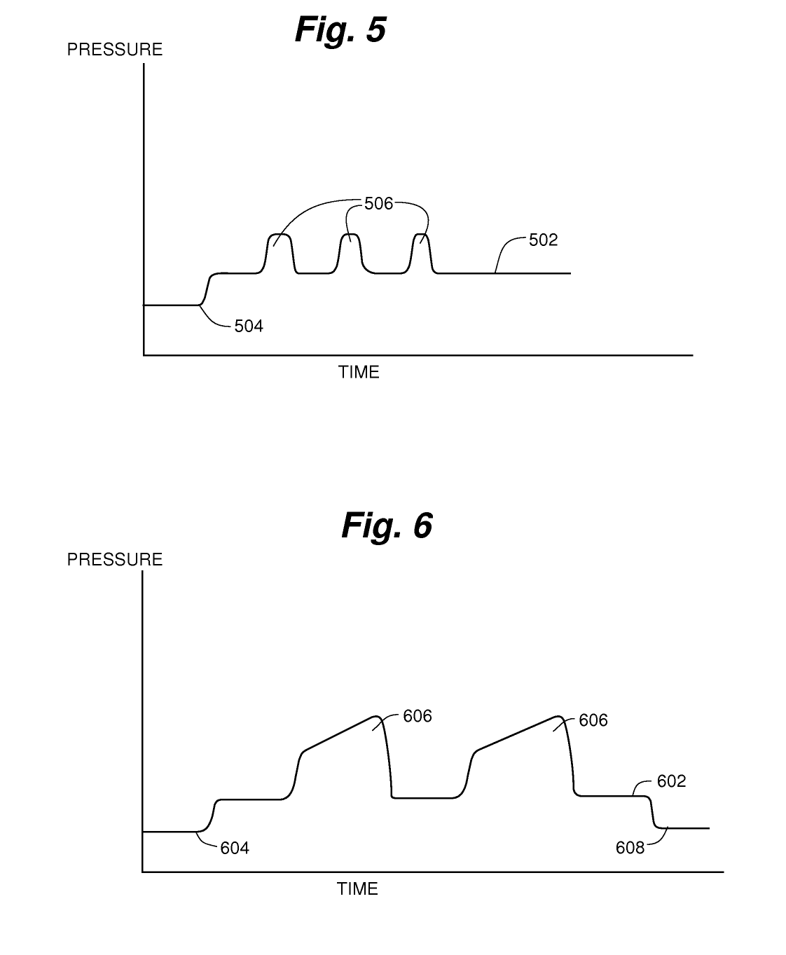

In another illustrative example (Scenario C), a medical practitioner or other appropriate agent can manipulate an infusion line in any appropriate manner in a way that can produce a predetermined pressure pattern resembling or similar to that illustrated in FIG. 5, which is a schematic graph of pressure vs. time. Reference numeral 502 represents a signal related to pressure in a fluid line. In Scenario C, the medical practitioner or agent can squeeze and hold the fluid line at 504 at a first location to substantially occlude the line, and then, while maintaining the occlusion of the line established at 504, repeatedly squeeze and release the infusion line at one or more other location(s), resulting in pulses 506. The one or more other location(s) at which the line is repeatedly squeezed and released can be located between the first location of the occlusion and the infusion pump, that is, upstream of the occlusion on a length of line downstream of the pump, or downstream of the occlusion on a length of line upstream of the pump. The creation of an occlusion at 504 can increase the amplitude of the pulses 506 by reducing the volume of the portion of line where the repeated squeezes act to increase the pressure in the line. The pressure pattern of FIG. 5 may represent a pressure pattern that would be produced when the manipulation of Scenario C is performed on a line attached to an idle pump. A downstream/(upstream) line attached to a running pump might see a pressure pattern with a gradually rising/(dropping) baseline pressure after the occlusion at 504, onto which the short-term pulses 506 can be added or overlaid. An interpretation/recognition algorithm can include recognition of such a gradually shifting baseline, or filtering of low-frequency information might reduce or eliminate the slowly-varying baseline signal component.

In another illustrative example (Scenario D), a medical practitioner or other agent can manipulate an infusion line to produce a predetermined pressure pattern resembling or similar to that illustrated in FIG. 6, which is a schematic graph of pressure vs. time. Reference numeral 602 represents a signal related to pressure in a fluid line. In Scenario D, the medical practitioner or other agent can squeeze and hold the fluid line at 604 at a first location to substantially occlude the line. While maintaining the occlusion of the line established at 604, the agent can then manipulate the line to produce ramped pulses 606. Each of ramped pulses 606 may be produced, for example, by pinching the line at a second pinch location between the first location of the occlusion and the infusion pump, sliding the second pinch location toward the infusion pump, then releasing the pinched portion. At 608, the squeezed portion at 604 that created the first occlusion can be released. It is envisioned that these manipulations can be performed by a medical practitioner or other agent with a hand or in any other suitable way. The present disclosure contemplates that manipulation of Scenario D and other scenarios also can be performed, for example, by machines, or as discussed further herein, by a medical practitioner or other agent manipulating a hand-operable tool. The particular predetermined pressure pattern of Scenario D is merely another example of a pattern, and is illustrative of the fact that further varieties of pressure patterns can be produced with distinctive features that may be amenable to machine recognition.

Returning to FIG. 2, infusion system 200 can include a tool 220 configured to create a predetermined pressure pattern in fluid line 204. Tool 220 can be configured to engage fluid line 204, and, when engaged with the fluid line, the tool can be configured to both occlude the fluid line and subsequently squeeze the fluid line. The actions of occluding and squeezing the fluid line 204 can produce a predetermined pressure pattern in the fluid line as described elsewhere herein. Tool 220 can include a clamp portion 222 configured to occlude fluid line 204, and one or more squeeze portions 224, where each of the one or more squeeze portions can be configured to squeeze the fluid line and thereby produce a pressure pulse in the fluid line. Tool 220 can include a grip 226 mechanically connected to clamp portion 222 and the one or more squeeze portions 224. Grip 226 can be configured and connected such that as the grip is actuated (for example, by squeezing by hand), clamp portion 222 can occlude fluid line 204 and then each of the squeeze portions 224 can squeeze the fluid line sequentially. Clamp portion 222 and/or squeeze portion(s) 224 can be directly connected to grip 226, or tool 220 can include intervening structures that connect the clamp and/or squeeze portions portion to the grip. Clamp portion 222, squeeze portion(s) 224, grip 226 and any intervening structures can be arranged and structured in any appropriate way, and can include, for example, shaping and material properties (e.g., elasticity) designed to result in engagement of the clamp portion 222 and squeeze portion(s) 224 with fluid line 204 in a desired manner, to result in a predetermined pressure pattern as tool 220 is actuated relative to fluid line 204. In some examples, tool 220 can be structured with all squeeze portions 224 disposed on the same side of clamp portion 222. Tool 220 can be engaged with fluid line 204 such that when the tool is actuated, squeeze portions 224 squeeze the fluid line between clamp portion 222 and infusion pump 202.

Tool 220 can be configured such that clamp 222 occludes, and then each of the one or more squeeze portions 224 squeeze, fluid line 204 sequentially as grip 226 is actuated in a continuous one-way motion, meaning that the actions can occur sequentially, for example, as the grip handles as illustrated in FIG. 2 are squeezed together progressively closer (“continuous one-way” meaning that the grip handles only are moved closer together, and not further apart, during the motion). In some examples, a tool such as tool 220 can be manipulated to produce a pre-determined pressure pattern in a motion that is not a continuous one-way motion. For example, if appropriately configured, tool 220 can be manipulable such that a medical practitioner or other agent could squeeze grip 226 sufficiently to occlude line 204 with clamp 222, squeeze further until at least one squeeze portion 224 squeezed the line to produce a pressure increase, relax the squeezing enough to disengage any number of squeeze portions from the line (but perhaps not enough to open the occlusion by the clamp, although the clamp could be disengaged also), then re-squeeze and relax the grip again to reengage the squeeze portion with the line to produce one or more further pressure pulses.

To assist a user in such manipulations, tool 220 can include a lock mechanism 228 (e.g., similar to those featured on some locking forceps) configured to reversibly lock after the clamp portion 222 occludes the fluid line 204 such that the fluid line remains occluded by the clamp portion. Lock mechanism 228 or another lock mechanism can be structured to reversibly lock after the one or more squeeze portions 224 squeeze the fluid line such that the fluid line 204 remains squeezed by the one or more squeeze portions. Tool 220 also can be structured such that it does not lock after the one or more squeeze portions 224 squeeze line 204, Such intentional non-locking may be desirable, for example, to permit a medical practitioner or other agent to squeeze the line multiple times, without locking interference, by repeatedly tightening and relaxing a grip on the tool. Note also that inclusion of a lock mechanism in a line squeezing tool can obviate the need to provide a separate line clamp for stopping fluid flow, although in some cases a simple line clamp can be provided in addition to a line squeezing tool.

Predetermined pressure patterns can exhibit any suitable temporal characteristics. A pressure pattern, whether produced through direct manual manipulation of a line, via a tool such as tool 220, or by another device or mechanism, need not necessarily adhere strictly to a particular timing pattern to be recognized as a predetermined pressure pattern. For example, time intervals between pulses 506 of signal 502 of FIG. 5 can vary, as can the durations of the pulses, while still being part of a recognizable predetermined pressure pattern. Such timing variations could result from variability of the process that produces the pattern, for example, a user may squeeze the grip 226 of a tool 220 more slowly or quickly from use to use. In some other examples, devices or mechanisms can be configured to produce precisely repeatable predetermined pressure patterns. For example, an electro-mechanical line-squeezing device could be provided to produce very consistent predetermined pressure patterns in lines. Predetermined pressure pattern detection/recognition/interpretation mechanisms and algorithms can be tailored to allow for greater or lesser degrees of consistency or variation in pressure patterns. Predetermined pressure patterns can include pressure variations having any suitable frequency components, and can include sub-sonic, acoustic, and/or ultra-sonic pressure variations. Any pressure patterns that are detectable by one or more sensors provided or associated with an infusion pump can be used. In some cases, a sensor used to measure pressure in a line to detect predetermined pressure patterns can sample at a rate of less than about 2000, 1000, 500, 100, 50, or 10 times per second (Hz).

It is contemplated that a line squeezing tool such as tool 220 could be provided with each fluid line such as lines 31-37 and/or lines 21-24 of FIG. 1, when, for example, a line is provided in a manufacturer's package for end use in an infusion administration set. In some instances, multiple tools can be provided on a single fluid line. For example, when a fluid line extends from an upstream reservoir, through an infusion pump, and to a downstream infusion site, line squeezing tools like tool 220 can be provided for portions of the fluid line both upstream and downstream from the pump. Tools provided with each fluid line can be configured to not be readily disengageable from the fluid line with which it is provided when the fluid line is deployed for patient therapy. For example, in some cases a line squeezing tool 220 can be configured such that the tool can be “slid” axially over a line (or equivalently, the line can be “threaded” through an aperture of the tool) during assembly of the infusion administration set, but when deployed for patient therapy, the tool can be blocked from being slid off one or both ends of the line, for example by a line connector affixed to a line end, other hardware, or the patient's body at the infusion site. Providing a tool that is not readily disengageable from the fluid line with which it is provided can help ensure that the tool is available and appropriately located when needed or desired. In some instances, a tool can be anchored or otherwise fixed at a particular longitudinal location along a line. In some other instances, a line squeezing tool such as tool 220 can be configured to be readily engaged with and disengaged from any of one or more fluid lines when the lines are deployed for use; as such, when engaged to any appropriate fluid line, the tool is capable of squeezing and occluding the line to produce a predetermined pressure pattern as described herein. In some cases, placement of a line squeezing tool that is readily engageable with a line can be facilitated by inclusion of features such as markings on a line indicating a suggested or preferred tool placement location and/or one or more hardware elements fixed with respect to the line that can mate or align with, or otherwise guide placement of, the tool with the line.

Tool 220 can assist clinicians by providing a relatively easy and reproducible way of intentionally creating/producing predetermined pressure patterns. Tool 220 can help transform relatively simple forces and motions (squeezing the grip 226 of the tool, a single time, or in some cases, multiple times) into more complex forces and motions that result in a predetermined pressure pattern that can be more distinctive and/or precise, possibly making the predetermined pressure pattern more readily machine-recognizable. Other aspects of using a tool are contemplated. In a primary aspect, tool 220 can be used to produce a predetermined pressure pattern in a line 204 to help identify the pump to which the line is connected. After this use, tool 220 can remain in a state for an indeterminate time interval where the line 204 is occluded thereby, particularly if the lock mechanism 228 locks tool 220 after it has produced the predetermined pressure pattern. Alternatively, a tool 220 without a lock mechanism can continue occluding a line, for example, if the tool grip remains manually squeezed. It is to be recognized, however, that in some instances it can be undesirable or problematic to maintain the occlusion of line 204 via tool 220 if fluid flow through the line is subsequently desired. Thus, in some examples, system 200 can include features to monitor for release of tool 220.

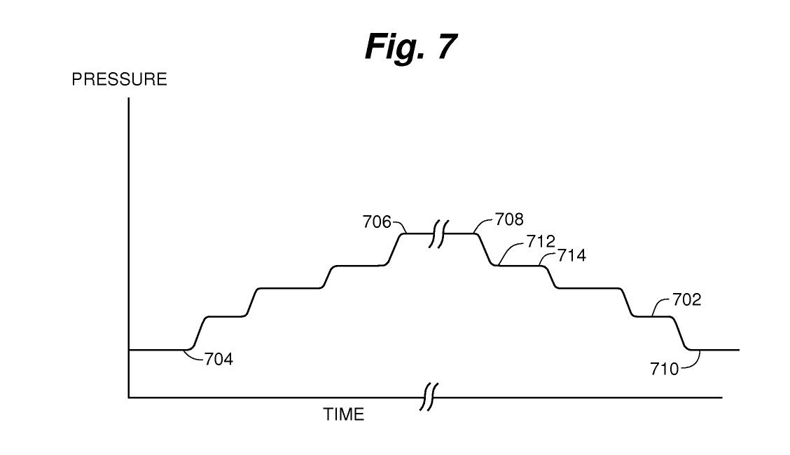

In an illustrative example (Scenario E), a medical practitioner or other agent can manipulate an infusion line with a line squeezing tool such as tool 220 to produce a predetermined pressure pattern. Subsequently, the tool can be released, and in a secondary aspect (relative to the primary aspect of producing a predetermined pressure pattern), the tool can produce a release pressure pattern associated with release of the tool from the line. FIG. 7 is a schematic graph of pressure vs. time in a fluid line that illustrates such a scenario. Reference numeral 702 represents a signal related to pressure in the fluid line. Upon actuating the tool, a predetermined pressure pattern can commence at 704 with occlusion of the line (following a pattern influenced at least in part by the configuration of the tool) and then end at 706 where the tool can complete its squeezing motion. At a later time 708 (which may be arbitrarily later than event 706, as indicated by the breaks in the graph and the time axis), the tool can be released and its motion reversed, resulting in a release pressure pattern associated with release of the tool that ends at 710 when the occlusion of the line by the tool can end.

By symmetry, the release pressure pattern can substantially mirror the predetermined pressure pattern, but this may not always be the case and is not required. For example (Scenario F), a tool could be configured with a lock mechanism that engages after the clamp portion and, for example, two of three squeeze portions engage the fluid line but before the third squeeze portion engages the fluid line. The predetermined pressure pattern can include pressure features resulting from engagement of the clamp with the fluid line, engagement of all three squeeze portions with the fluid line, and disengagement of the third squeeze portion from the fluid line. With release of the hold on the tool, the lock mechanism can maintain engagement of the clamp portion and first two squeeze portions with the fluid line. Upon release of the lock mechanism, a release pressure pattern can result as the second and first squeeze portions and the clamp portion disengage from the fluid line in sequence. With reference to FIG. 7, in Scenario E the predetermined pressure pattern can end at 712 rather than 706 (disregarding the break in the graph between 706 and 708), and the release pressure pattern can be observed from 714 to 710 (with an arbitrary passage of time possible between 712 and 714).

Similarly as with the detection of predetermined pressure patterns, one or more components of an infusion pump such as pump 202 of FIG. 2 (and/or possibly external devices) can be configured to monitor for and detect a release pressure pattern associated with release of a tool used to produce predetermined pressure patterns. Information regarding detection (or lack of detection) of a release pressure pattern can be used in any suitable manner. For example, a controller such as controller 216 or any other component or system can continue to maintain an indication of detection of a predetermined pressure pattern if a release pressure pattern has not been detected. This can serve to indicate to and/or remind a medical practitioner or another that the line attached to a pump is occluded and that the pump will not operate properly or as if the line was not occluded. In another example, a controller or any other component or system can annunciate an alarm if a pre-determined condition is met (such as occurrence of an attempt to start medicament delivery, or passage of a pre-determined time interval, etc.) and the release pressure pattern has not been detected.

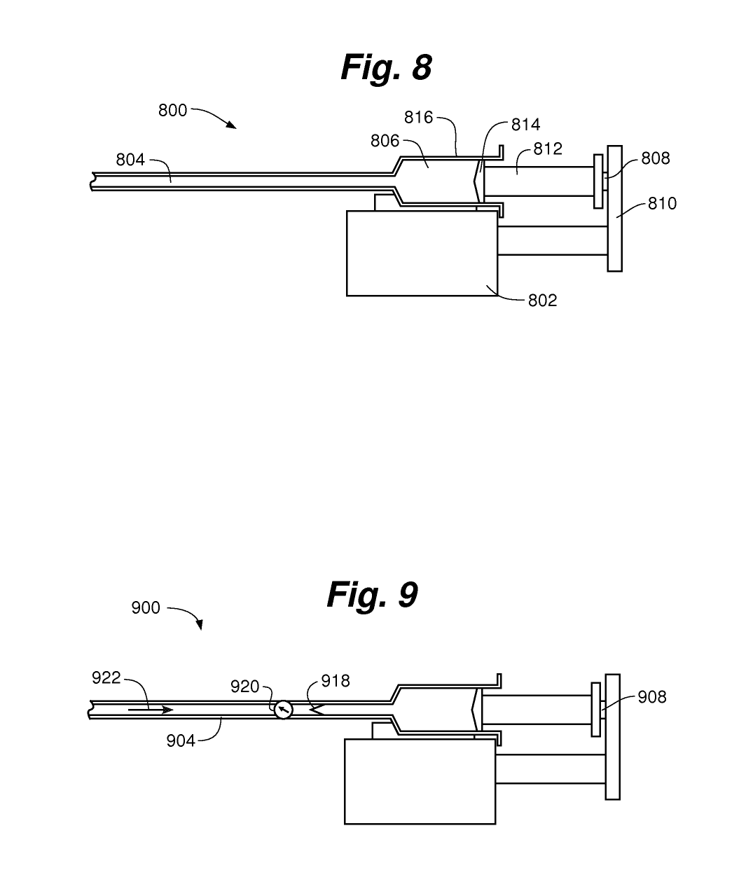

In some systems, it is contemplated that detection of predetermined pressure patterns for line identification can be performed effectively with pump components that also are purposed with other tasks, such as pressure sensors for occlusion detection. For example, in the system 200 of FIG. 2, detector 212 can sense pressure for occlusion detection and for predetermined pressure pattern recognition. In some configurations, however, while it may be possible to employ an existing occlusion detector for detection of predetermined pressure patterns, judicious placement of an additional pressure sensor can provide an improved pressure signal. FIG. 8 is a schematic diagram of portions of a syringe infusion pump system 800 that includes a syringe pump 802 that acts to deliver medicament into a fluid line 804 from a syringe 806 (the relative diameters of the fluid line and syringe are not to scale, nor are other aspects of this schematic drawing). Syringe pump 802 can include a downstream occlusion sensor 808 at a point of contact between plunger driver 810 and plunger 812. When a downstream occlusion is present and the pump is running, sensor 808 can sense an increasing and greater than nominal contact force between the plunger driver 810 and plunger 812, which can trigger an occlusion alarm. While this arrangement may be suitable for occlusion detection, greater sensitivity may be desired for detection of predetermined pressure patterns, which may exhibit relatively smaller variations in pressure amplitude, and which may vary at higher frequencies, than signals indicating conventional occlusions. Aspects of the arrangement of FIG. 8 may contribute to a substantial degree of undesirable compliance as pressure is transmitted upstream back toward the downstream occlusion sensor 808, such as may be caused by one or more physical interactions in a relatively large volume of the syringe reservoir and friction between plunger piston 814 and barrel 816 of the syringe.

FIG. 9 is a schematic diagram of portions of a syringe infusion pump system 900 similar to system 800 of FIG. 8 that includes additional features that can improve monitoring of pressure in fluid line 904 as compared with system 800. Fluid line 904 includes a valve 918 that can be a one-way valve (for example, a leaf valve) configured to prevent upstream flow back toward the syringe. A pressure sensor 920 configured to measure pressure in fluid line 904 is disposed on the downstream side of valve 918. Accordingly, when a predetermined pressure pattern is produced in fluid line 904 by manipulation of the line downstream from valve 918 (and is thus at least potentially detectable at sensor 920), closure of valve 918 upstream of sensor 920 can prevent fluid flow and the pressure pattern from propagating upstream of the valve, removing compliance in the syringe as a factor that many diminish sensitivity to pressure signals.

While the present disclosure provides multiple methods of producing predetermined pressure patterns that involve occluding and/or squeezing lines, other ways of producing predetermined pressure patterns are contemplated. For example, pressure in an upstream line between an external reservoir and a pump can be manipulated by varying the height of the reservoir; raising/lowering the reservoir generally would increase/decrease the pressure in the line. Other manipulations of reservoir and/or lines are contemplated that may vary the pressure measured at a sensor in a predictable way in order to produce predetermined pressure patterns for line identification.

The disclosure should not be considered limited to the particular examples described herein, but rather should be understood to cover all aspects of the disclosure and equivalents thereof. Various modifications, equivalent processes, as well as numerous structures to which the disclosure can be applicable will be readily apparent to those of skill in the art upon review of the instant specification.

1. A method for identifying to which one of a plurality of infusion pumps one of a plurality of fluid lines is coupled, the method comprising:
intentionally producing a predetermined pressure pattern in one of a plurality of fluid lines;
detecting the predetermined pressure pattern by way of a sensor of one of a plurality of infusion pumps; and
indicating detection of the predetermined pressure pattern in the one of the plurality of fluid lines, thereby indicating the one of the plurality of infusion pumps to which the one of the plurality of fluid lines is coupled.

2. The method of claim 1, wherein intentionally producing the predetermined pressure pattern includes:
occluding the one of the plurality of fluid lines at a first location; and
while occluded at the first location, squeezing the one of the plurality of fluid lines.

3. The method of claim 2, wherein the squeezing of the one of the plurality of fluid lines includes squeezing between the first location and the one of the plurality of infusion pumps.

4. The method of claim 2, wherein the squeezing includes squeezing the one of the plurality of fluid lines at least two times.

5. The method of claim 2, wherein the intentionally producing the predetermined pressure pattern includes actuating a tool configured to perform the occluding and the squeezing.

6. The method of claim 5, wherein the tool is configured to be readily engaged with and disengaged from any of the plurality of fluid lines when the lines of the plurality of fluid lines are deployed for use; and wherein the tool, when engaged, is capable of performing the occluding and the squeezing.

7. The method of claim 5, wherein the tool is configured to be not readily disengageable from the one of the plurality of fluid lines when the fluid line is deployed for use.

8. The method of claim 5, wherein the tool is structured to perform the occluding and the squeezing sequentially as handles of the tool are progressively brought together.

9. The method of claim 5, wherein the tool is configured to reversibly lock after occluding the one of the plurality of fluid lines.

10. The method of claim 5, wherein the tool is configured to reversibly lock after the occluding and the squeezing of the one of the plurality of fluid lines.

11. The method of claim 5, wherein the tool is configured not to lock after the squeezing of the one of the plurality of fluid lines, thereby permitting squeezing of the line multiple times without locking interference by repeatedly tightening and relaxing a hold on the tool.

12. The method of claim 5, further comprising the step of monitoring for a release pressure pattern associated with release of the tool.

13. The method of claim 12, further comprising the step of maintaining an indication of detection of the predetermined pressure pattern if the release pressure pattern has not been detected.

14. The method of claim 12, further comprising the step of annunciating an alarm if a pre-determined condition is met and the release pressure pattern has not been detected.

15. The method of claim 1, wherein the indicator is disposed in or on the one of the plurality of infusion pumps.

16. The method of claim 1, wherein the sensor is configured to perform upstream occlusion detection.

17. The method of claim 1, wherein the sensor is configured to perform downstream occlusion detection.

18. The method of claim 1, wherein the sensor is disposed downstream relative to a valve of the one of the plurality of fluid lines.

19. The method of claim 1, wherein a predetermined pressure pattern is a relationship of pressure vs. time in the one of the plurality of fluid lines that is attributable to intentionally producing the relationship, with such intentional production being more complex than creation of a single occlusion of the one of the plurality of fluid lines.

20. A system for confirming that a fluid line is connected to an infusion pump, comprising:
a fluid line;
an infusion pump configured to pump fluid in the fluid line, the infusion pump including:
a sensor configured to detect pressure of fluid in the fluid line and output information related to the pressure of the fluid;
a controller operatively coupled to the sensor; and
an indicator operatively coupled to the controller; and
a tool configured to engage the fluid line, and, when engaged with the fluid line, configured to both occlude the fluid line and subsequently squeeze the fluid line, wherein the actions of occluding and squeezing the fluid line produce a predetermined pressure pattern in the fluid line,
wherein the controller is programmed and configured to:
receive the information related to the pressure of the fluid;
interpret the information related to the pressure of the fluid to recognize the predetermined pressure pattern; and
if the predetermined pressure pattern is recognized, provide an indication via the indicator.

21. An infusion pump, comprising:
a pumping mechanism configured to supply a fluid medicament from a reservoir to a patient via a line;
a sensor configured to measure pressure of the fluid medicament in the line and output information related to the pressure;
a controller operatively coupled to the sensor; and
an indicator operatively coupled to the controller,
wherein the controller is programmed and configured to:
receive the information related to the pressure; and
interpret the information related to the pressure to recognize any of a group of one or more predetermined pressure patterns; and
if any of the group of one or more predetermined pressure patterns is recognized, provide an indication via the indicator.

22. A device for creating a predetermined pressure pattern in a fluid line, the device comprising:
a clamp portion configured to occlude the fluid line;
one or more squeeze portions, each of the one or more squeeze portions configured to squeeze the fluid line and thereby produce a pressure pulse in the fluid line; and
a grip mechanically connected to the clamp portion and the one or more squeeze portions, the grip configured and connected such that as the grip is actuated, the clamp occludes the fluid line and then each of the one or more squeeze portions squeeze the fluid line sequentially.

23. The device of claim 22, wherein the clamp occludes, and then each of the one or more squeeze portions squeeze, the fluid line sequentially as the grip is actuated in a continuous one-way motion.

24. The device of claim 22, further comprising a lock mechanism configured to reversibly lock after the clamp portion occludes the fluid line such that the fluid line remains occluded by the clamp portion.

25. The device of claim 22, further comprising a lock mechanism configured to reversibly lock after the one or more squeeze portions squeeze the fluid line such that the fluid line remains squeezed by the one or more squeeze portions.

26. The device of claim 23, wherein the device is structured such that it does not necessarily lock after the one or more squeeze portions squeeze the fluid line.

27. A method for identifying an infusion pump to which one of a plurality of fluid lines is coupled, the method comprising:
intentionally producing a predetermined pressure pattern in one of a plurality of fluid lines;
detecting the predetermined pressure pattern by way of a sensor of an infusion pump; and
indicating detection of the predetermined pressure pattern in the one of the plurality of fluid lines, thereby indicating that the one of the plurality of fluid lines is coupled to the infusion pump.
